(19) United States
(12) Patent Application Publication
(10) Pub. No.: US 2024/0009177 A1
(43) Pub. Date: Jan. 11, 2024

(54) COMBINATION COMPOSITIONS HAVING ANTI-VIRAL ACTIVITIES AND USES THEREOF

(71) Applicant: RAMOT AT TEL-AVIV UNIVERSITY LTD., Tel Aviv (IL)

(72) Inventors: Dor ZAGURI, Tel Aviv (IL); Topaz KREISER-SHEM-TOV, Tel Aviv (IL); Ehud GAZIT, Tel Aviv (IL); Daniel SEGAL, Tel Aviv (IL); Josef MOGRABI, Tel Aviv (IL); Eran BACHARACH, Tel Aviv (IL)

(21) Appl. No.: 18/245,970

(22) PCT Filed: Sep. 19, 2021
(86) PCT No.: PCT/IL2021/051134

§ 371 Date: Mar. 20, 2023

Related U.S. Application Data
(60) Provisional application No. 63/080,797, filed on Sep. 21, 2020.

(51) Int. Cl. A61K 31/4375; A61K 31/352; A61K 33/34; A61K 31/315; A61P 31/16

(52) U.S. Cl. CPC A61K 31/4375; A61K 31/352; A61K 33/34; A61K 31/315; A61P 31/16

(57) ABSTRACT

Combination compositions exhibiting anti-viral activities are provided, which are based on unique combinations of polyphenol zinc ionophores. Exemplary compositions comprise combinations of flavonoid zinc ionophores selected from EGCG, quercetin and taxifolin, preferably further comprising a zinc compound and a copper compound. The compositions preferably contain additional ingredients to further enhance the anti-viral effect of the composition and/or to provide additional benefits.

### FIELD OF THE INVENTION

The present invention relates to compositions exhibiting anti-viral activities comprising combinations of active ingredients. More particularly, the compositions are based on zinc ionophores, such as epigallocatechin 3-gallate (EGCG), quercetin and taxifolin (dihydroquercetin), combined with zinc and additional ingredients that enhance the anti-viral effect of the compositions and/or provide additional benefits. The compositions are particularly useful for restricting and treating viral infections.

### BACKGROUND OF THE INVENTION

A virus is an infectious agent that replicates inside living cells of an organism. Viruses infect all types of life forms, from animals and plants to microorganisms. Presumably, there are millions of types of viruses in the environment, with more than 6,000 virus species described in detail. When infected, a host cell is forced to rapidly produce thousands of identical copies of the original virus. Viruses spread in many ways: e.g. through virus-bearing organisms (vectors), by coughing and sneezing, by the fecal—oral route (passed by hand-to-mouth contact or in food or water), through sexual contact and by exposure to infected blood.

Influenza is an infectious disease caused by influenza viruses. Influenza viruses comprise single-stranded RNA segments. The most common symptoms include: high fever, runny nose, sore throat, myalgias, joint pain, headache, coughing, and fatigue. Complications of influenza may include viral pneumonia, secondary bacterial pneumonia, sinus infections, and worsening of previous health problems such as asthma or heart failure.

Influenza causes significant loss of workdays, human suffering, and mortality. Influenza spreads around the world in yearly outbreaks mainly during the winter of the northern and southern hemispheres, resulting in about three to five million cases of severe illness and about 290,000 to 650,000 deaths. Although the number of cases of influenza can vary widely between years, approximately 36,000 deaths and more than 200,000 hospitalizations are directly associated with influenza every year in the United States.

Coronaviruses (CoVs) are the largest group of viruses belonging to the Nidovirales order. Coronaviruses are associated with illness from the common cold to more severe conditions such as Severe Acute Respiratory Syndrome (SARS-CoV) and Middle East Respiratory Syndrome (MERS-CoV). Severe acute respiratory syndrome coronavirus 2 (SARS-CoV-2) is an enveloped positive-sense, single-stranded RNA coronavirus which belongs to the betacoronavirus genera. The virus SARS-CoV-2 causes the coronavirus disease 2019 (COVID-19). Coronaviruses are zoonotic, meaning they are transmitted between animals and people. Common signs of coronavirus infection include respiratory symptoms, fever, coughing, shortness of breath and breathing difficulties. COVID-19 symptoms also include hypoxia, anosmia, and blood clots. High concentrations of cytokines were recorded in plasma of critically ill patients suffering from COVID-19. In more severe cases, infection can cause pneumonia, respiratory inflammation, acute respiratory distress syndrome (ARDS), kidney failure, sepsis, cytokine storm syndrome (CSS) and death.

The COVID-19 pandemic caused by SARS-CoV-2 has spread globally and is expected to infect many millions of people worldwide. To date, there are no specific medicines for COVID-19. Hence, finding a solution for this pandemic is essential for human health and critical for global economy.

Zinc supplementation was suggested to potentially reduce the risk of SARS-CoV-2 infections and shorten the duration and severity of illness (Arentz et al., Adv Integr Med., Zinc for the prevention and treatment of SARS-CoV-2 and other acute viral respiratory infections: a rapid review, available online 1 Aug. 2020; Mayor-Ibarguren et al. Front. Immunol. 2020. 11:1736; Wessels et al., Front. Immunol. 2020. 11:1712; Skalny et al., Int J Mol Med. 2020. 46:17-26).

Treating patients suffering from the coronavirus disease with a combination of zinc and chloroquine or hydroxychloroquine has been postulated to reduce the effects of the virus on the affected patients and improving clinical trials outcome compared to chloroquine or hydroxychloroquine without zinc (shittu et al., Le Infezioni in Medicina. 2020. 28(2):192-197). However, hydroxychloroquine is associated with severe adverse effects. Specifically, hydroxychloroquine was found to cause mainly cardiovascular adverse effects in COVID-19 patients.

Zn²⁺ and the zinc-ionophore pyrithione (PT) were shown to inhibit coronavirus and arterivirus RNA polymerase activity and block virus replication (te Velthuis et al., PLoS Pathog. 2010. 6(11): e1001176.)

Natural remedies were postulated to be effective for treating COVID-19 (Park J., Apr. 7, 2020, COVID-19 Alternative Medicine? Part-1: Supplement with Zinc, Quercetin & Epigallocatechin Gallate (EGCG)).

European Patent No. EP1556021 describes pharmaceutical compositions comprising flavonoids and menthol for treating common cold or similar conditions.

U.S. Pat. No. 8,003,688 describes a pharmaceutical composition, effective in vivo as an antiviral agent, comprising a pharmaceutically effective amount of at least one hydroxyethylrutoside or pharmaceutically acceptable salt thereof, said hydroxyetheylrutosides or salts thereof including troxerutin or a pharmaceutically acceptable salt thereof, said amount being antivirally effective in vivo, and an amount of zinc gluconate effective in vivo to cause the in vivo antiviral effect of the composition to exceed that of the hydroxyethylrutoside(s), or salts thereof, of the composition, wherein said composition is suitable for topical administration to the mucosal membrane of the oral cavity, and is not a solution, unless such solution is a spray.

International Patent Publication No. WO2017/029674 discloses the use of a combination of phytoene and phytofluene and compositions comprising same for delaying viral infection.

There is a need for safe compositions having anti-viral properties that can be consumed for prolonged time periods to reduce viral diseases, particularly respiratory viral diseases associated with winter sickness, that will also be useful for the treatment of such viral diseases. In particular, as currently there are no specific drugs to treat COVID-19, it would be highly advantageous to have a safe prevention method, based on ingredients that can be safely taken by people for prolonged periods of time, and that may also be beneficial in the treatment of patients. Such a method may benefit the entire community, by reducing loss of workdays, hospital workload, and more.

### SUMMARY OF THE INVENTION

The present invention provides combination compositions exhibiting anti-viral activities. The compositions of the present invention are based on unique combinations of flavonoids and other polyphenolic zinc ionophores. In some embodiments, compositions comprising combinations of flavonoid zinc ionophores, for example combinations of zinc ionophores selected from EGCG, quercetin and taxifolin, are provided, preferably further comprising a zinc compound and a copper compound. Without being bound by any particular theory of a mechanism of action, it is contemplated that such combinations increase intracellular zinc concentration (Zn 2+), which impairs the replication of RNA viruses. The compositions of the present invention are useful for preventing viral infections pre and/or post-exposure. According to some embodiments, the compositions of the present invention are useful for treating viral infections. In some embodiments, combinations of zinc ionophores at particularly effective amounts and/or proportions are provided. In additional embodiments, the compositions of the present invention contain additional ingredients combined with the zinc ionophores and zinc and copper compounds, to further enhance the anti-viral effect of the composition and/or provide additional benefits such as reduction in the severity of symptoms upon infection, reducing the risk of complications upon infection, and/or decreasing the ability of virus carriers to infect others. In some particular embodiments, the compositions of the present invention contain at least one phenol peroxisome proliferator-activated receptor (PPAR) agonist, for example, naringenin and/or berberine.

The combinations provided by the present invention are based on readily available ingredients, regarded as GRAS (Generally Recognized As Safe) in the US or being legally sold as dietary supplements in many other countries, that were found to exhibit a remarkable anti-viral activity when combined together. More particularly, as exemplified hereinbelow, combinations according to the present invention effectively inhibited viral infection and reduced viral load in several cell types including human lung cells infected with a variety of RNA viruses in vitro. The RNA viruses included three different viruses that cause respiratory infections, namely, an influenza A virus, a betacoronavirus and a metapneumovirus. Without being bound by any particular theory of a mechanism of action, the reduced intracellular viral load may be achieved by inhibiting or preventing intracellular virus replication at least in part via an increase of intracellular zinc concentration (Zn 2+). The compositions of the present invention are therefore not dependent upon inhibition of a specific receptor or a specific entry mechanism of a virus, and thus are useful against a variety of viruses, particularly RNA viruses, such as RNA viruses associated with winter respiratory infections. Exemplary viruses include an influenza virus, a respiratory syncytial virus (RSV), a metapneumovirus (MPV) and a betacoronavirus.

As exemplified hereinbelow, the combinations according to the present invention showed significant inhibition of viral infection and were more effective than each compound alone. Most compounds did not show any beneficial effect, or only showed a moderate effect. In some exemplary embodiments, combinations of taxifolin with several compounds were found to improve the effect achieved by each compound alone.

Surprisingly, not only that combinations according to the present invention show an effective inhibition of the virus, in some embodiments the inhibitory anti-viral effect exhibited by combinations according to the present invention is superior to that of the commercially available medication hydroxychloroquine and superior to hydroxychloroquine together with zinc compound. The medication hydroxychloroquine was proposed as anti-viral agent for the treatment of COVID-19, and the FDA even issued Emergency Use Authorization (EUA) for hydroxychloroquine sulfate to treat COVID-19 patients; the EUA was later revoked. It appeared that hydroxychloroquine is associated with severe adverse effects. Specifically, hydroxychloroquine causes mainly cardiovascular adverse effects in COVID-19 patients. Advantageously, as noted above, the ingredients in the compositions of the present invention are designated as GRAS in the US and thus can be consumed by people for extended periods of time. As a further advantage, the use of different types of zinc ionophores has the potential to affect a broad range of cell types since not all zinc ionophores are equally effective in all human cell types, tissues and organs. In some embodiments of the present invention, the combinations disclosed herein provide a synergistic anti-viral effect.

In some embodiments, the compositions according to the present invention are dietary supplement compositions. In additional embodiments, the compositions are pharmaceutical compositions. Combinations according to the present invention are useful in inhibiting viral infection and reducing viral load in cells infected with viruses, e.g., RNA viruses. Thus, in some embodiments, the compositions of the present invention are useful for preventing and treating infection by an RNA virus. In some particular embodiments, the virus is an influenza virus. In additional particular embodiments, the virus is human metapneumovirus (hMPV). In some other particular embodiments, the virus is a coronavirus. In additional particular embodiments, the virus is SARS-CoV-2.

In some embodiments, the compositions of the present invention are useful for preventing infection by the virus. In additional embodiments, the compositions of the present invention are useful for treating virus infections and for reducing severity of the disease upon infection. In additional embodiments, the compositions of the present invention are useful for decreasing the ability of virus carriers to infect others.

According to one aspect, the present invention provides a composition for human consumption comprising:
(a) at least two flavonoid zinc ionophores selected from the group consisting of: taxifolin, quercetin and epigallocatechin gallate (EGCG);
(b) at least one zinc compound; and
(c) at least one copper compound, optionally further comprising at least one phenol peroxisome proliferator-activated receptor (PPAR) agonist.

In some embodiments, the composition comprises at least one phenol peroxisome proliferator-activated receptor (PPAR) agonist. In some embodiments, the at least one PPAR agonist is selected from the group consisting of naringenin, naringin, mangiferin, berberine, resveratrol, chlorogenic acid and baicalein. In some particular embodiments, the composition comprises the PPAR agonist naringenin. In additional particular embodiments, the composition comprises the PPAR agonist berberine.

In some embodiments, the composition comprises: taxifolin and at least one additional flavonoid zinc ionophore selected from quercetin and EGCG; at least one zinc compound; and at least one copper compound.

In some embodiments, the composition comprises the flavonoid zinc ionophores taxifolin, quercetin and EGCG; a zinc compound; and a copper compound. In some embodiments, the composition further comprises naringenin. In some embodiments, the composition further comprises berberine.

In some embodiments, the zinc compound in compositions according to the present invention is zinc picolinate.

In some embodiments, the copper compound in compositions according to the present invention is copper sulfate.

In some embodiments, the composition is a dietary supplement composition. In other embodiments, the composition is a pharmaceutical composition. In some embodiments, the composition is for oral consumption. A composition according to the present invention may be in a form selected from the group consisting of a pill, a tablet, a caplet, a capsule, a softgel a powder and a lozenge. Additional suitable forms include sublingual and/or buccal administration compositions, sachets, bars, in yogurt and in functional foods and functional drinks. A composition according to the present invention may be formulated for administration as a nasal spray. A composition according to the present invention may also be formulated for administration via routs such as intravenous (IV), subcutaneous (SC), intramuscular (IM) and intraperitoneal (IP), or using inhalers (including disks) and inhalation vaporizers. Each of the aforementioned possibilities represents a separate embodiment of the present invention.

In some embodiments, the composition is for providing an anti-viral benefit/an anti-viral support to a subject, particularly a human subject.

In some embodiments, the compositions of the present invention are for use in preventing viral infection pre-exposure and post-exposure. Each possibility represents a separate embodiment of the present invention. In additional embodiments, the compositions of the present invention are for use in reducing severity of the disease upon infection. In yet additional embodiments, the compositions of the present invention are for use in decreasing the ability of virus carriers to infect others. In yet additional embodiments, the compositions of the present invention are for use in treating a viral infection.

In some embodiments, the compositions reduce viral load in the host. In additional embodiments, the compositions inhibit viral replication in the host.

In some embodiments, the virus is an RNA virus. In some embodiments, the virus is an influenza virus. In additional embodiments, the virus is a coronavirus. In additional particular embodiments, the virus is a betacoronavirus. In additional particular embodiments, the betacoronavirus is SARS-CoV-2.

According to a further aspect, the present invention provides a method for providing an anti-viral benefit/an anti-viral support to a subject, particularly a human subject, the method comprising administering to the subject a composition according to the present invention.

In some embodiments, there is provided a method for providing an anti-viral benefit and/or an anti-viral support to a subject, the method comprising co-administering to the subject: (a) at least two flavonoid zinc ionophores selected from the group consisting of: taxifolin, quercetin and epigallocatechin gallate (EGCG); (b) at least one zinc compound; and (c) at least one copper compound, optionally further comprising co-administering at least one phenol peroxisome proliferator-activated receptor (PPAR) agonist.

In some embodiments, the method comprises co-administering at least one phenol peroxisome proliferator-activated receptor (PPAR) agonist selected from the group consisting of naringenin, naringin, mangiferin, berberine, resveratrol, chlorogenic acid and baicalein. In some particular embodiments, the method comprises co-administering naringenin.

According to a further aspect, the present invention provides a method for preventing a viral infection in a subject, particularly a human subject, the method comprising administering to the subject a composition according to the present invention.

In some embodiments, there is provided a method for preventing a viral infection in a subject, the method comprising co-administering to the subject: (a) at least two flavonoid zinc ionophores selected from the group consisting of: taxifolin, quercetin and epigallocatechin gallate (EGCG); (b) at least one zinc compound; and (c) at least one copper compound, optionally further comprising co-administering at least one phenol peroxisome proliferator-activated receptor (PPAR) agonist.

In some embodiments, the method further comprises co-administering at least one phenol peroxisome proliferator-activated receptor (PPAR) agonist selected from the group consisting of naringenin, naringin, mangiferin, berberine, resveratrol, chlorogenic acid and baicalein. In some particular embodiments, the method comprises co-administering naringenin. In some particular embodiments, the method further comprises co-administering berberine.

According to a further aspect, the present invention provides a method for treating a viral infection in a subject, particularly a human subject, the method comprising administering to the subject a composition according to the present invention.

In some embodiments, there is provided a method for treating a viral infection in a subject, the method comprising co-administering to the subject: (a) at least two flavonoid zinc ionophores selected from the group consisting of: taxifolin, quercetin and epigallocatechin gallate (EGCG); (b) at least one zinc compound; and (c) at least one copper compound, optionally further comprising co-administering at least one phenol peroxisome proliferator-activated receptor (PPAR) agonist.

In some embodiments, the method further comprises co-administering at least one phenol peroxisome proliferator-activated receptor (PPAR) agonist selected from the group consisting of naringenin, naringin, mangiferin, berberine, resveratrol, chlorogenic acid and baicalein. In some particular embodiments, the method further comprises co-administering naringenin. In some particular embodiments, the method further comprises co-administering berberine.

In some embodiments, the composition is administered daily for a period of time ranging, for example, from 1-6 months, including each value within the range. In additional embodiments, the composition is for daily administration for longer periods of time. For example, in some embodiments, the composition is administered daily for 6-24 months, including each value within the range.

In additional embodiments, the composition is administered for shorter periods of time, for example, for several days up to 1 month. For example, in some embodiments, the composition is administered for 4-10 days, or 10-30 days, including each value within the range. Each possibility represents a separate embodiment of the present invention.

The subject is typically a human subject. The subject may also be a non-human animal. In some embodiments, the subject is not infected with a virus, and the composition is administered for preventing infection. In some particular embodiments, the subject is a subject exposed to the virus, or suspected of being exposed to the virus. In other embodiments, the subject is infected with a virus, and the composition is administered for treating the viral infection and/or reducing the probability of infection of others by the subject. In some embodiments, the subject is a subject at risk for complications of viral infections. High-risk subjects may include, for example, subjects over 60 years old and/or subjects with one or more background diseases such as a lung disease, a heart disease, diabetes (type I or type II), high blood pressure, overweight or obesity, malnutrition and malabsorption. High-risk subjects may also include immunocompromised subjects, such as subjects suffering from a congenital disorder of the immune system, subjects suffering from an acquired disorder of the immune system or subjects immunocompromised due to certain medications.

Other objects, features and advantages of the present invention will become clear from the following description, examples and drawings.

### BRIEF DESCRIPTION OF THE FIGURES

FIGS. 1A-1C. Measurements of viral infection. A549 human epithelial carcinoma lung cells seeded the day before in a 24-well plate (0.24×10⁶ cells per well) were treated for 4 hours with the indicated compositions (Zinc picolinate (ZnPico) (40 μM)+Taxifolin (Taxi) (40 μM)+epigallocatechin gallate (EGCG) (40 ZnPico (40 μM)+Quercetin (40 μM)+EGCG (40 μM) ZnPico (40 μM)+HCQ (20 μM), HCQ (20 Taxifolin (40 μM) or medium+PBS+DMSO; all in total of 300 μL); and subsequently infected with influenza A virus, using calculated multiplicity of infection (MOI) of ˜0.5 (FIG. 1A), 0.1 (FIG. 1B) and 0.05 (FIG. 1C) and incubated for 1 hour in the presence of the virus. The virus-containing medium was then replaced with a fresh medium containing only the indicated compositions, and the cells were incubated for additional 24 hours, after which viral infections was quantitatively assessed.

FIG. 2 is a graph showing free zinc levels quantification of lysates of A549 lung cells treated with different compositions (ZnPico (40 μM)+Taxifolin (Taxi) (40 μM)+EGCG (40 ZnPico (40 μM)+Quercetin (40 μM)+EGCG (40 ZnPico (40 μM)+HCQ (20 ZnPico (40 μM)+medium+DMSO or medium+DMSO+PBS). Zinc levels are shown as % normalized with respect to control (cells treated with medium+DMSO+PB S).

FIG. 3. Toxicity evaluation using MTT cell viability assay. A549 human epithelial carcinoma lung cells seeded the day before in a 96-well plate (0.45×10⁵ cells per well) were treated for 48 hours with the indicated compositions. The results are shown as % viability normalized with respect to control (untreated cells).

FIG. 4A-4B. Viral infection inhibition in A549 cells (FIG. 4A) and SH-SYSY cells (FIG. 4B) infected with influenza A PR8 (H1N1) virus and treated with the indicated compositions. “Composition #1” is a 5-compound composition containing: zinc picolinate (40 μM)+copper sulfate (10 μM)+quercetin (40 μM)+EGCG (40 μM)+taxifolin (40 μM).

FIG. 5. Viral infection inhibition in A549 cells infected with PR8 influenza A (H1N1) virus and treated with the indicated compositions. “Composition #2” is a 6-compound composition containing: zinc picolinate (40 μM)+copper sulfate (10 μM)+quercetin (40 μM)+EGCG (40 μM)+taxifolin (40 μM)+naringenin (10 μM).

FIG. 6A-6B. Viral infection inhibition in A549 cells (FIG. 6A) and H1299 cells (FIG. 6B) infected with human coronavirus OC43 (HCoV-OC43) and treated with the indicated compositions. “Composition #1” is a 5-compound composition containing: zinc picolinate (40 μM)+copper sulfate (10 μM)+quercetin (40 μM)+EGCG (40 μM)+taxifolin (40 μM). “Composition #2” is a 6-compound composition containing: zinc picolinate (40 μM)+copper sulfate (10 μM)+quercetin (40 μM)+EGCG (40 μM)+taxifolin (40 μM)+naringenin (10 μM).

FIG. 7. Viral infection inhibition in A549 cells infected with human coronavirus (HCoV-OC43) and treated with the indicated compositions. “Composition #2” is a 6-compound composition containing: zinc picolinate (40 μM)+copper sulfate (10 μM)+quercetin (40 μM)+EGCG (40 μM)+taxifolin (40 μM)+naringenin (10 μM).

FIG. 8. Viral infection inhibition in Vero cells infected with human metapneumovirus (hMPV) and treated with the indicated compositions. “Composition #1” is a 5-compound composition containing: zinc picolinate (40 μM)+copper sulfate (10 μM)+quercetin (40 μM)+EGCG (40 μM)+taxifolin (40 μM).

FIG. 9A-9B. Toxicity evaluation using MTT cell viability assay of varying doses of compounds according to the present invention in A549 cells (FIG. 9A) and H1299 cells (FIG. 9B).

FIG. 10. Toxicity evaluation using MTT cell viability assay of compounds according to the present invention and a combination according to the present invention in Vero cells and SH-SYSY cells. “Composition #”1 is a 5-compound composition of: zinc picolinate (40 μM)+copper sulfate (10 μM)+quercetin (40 μM)+EGCG (40 μM)+taxifolin (40 μM).

FIG. 11. Toxicity evaluation using MTT cell viability assay of combinations according to the present invention in A549 cells. “Composition #”1 is a 5-compound composition of: zinc picolinate (40 μM)+copper sulfate (10 μM)+quercetin (40 μM)+EGCG (40 μM)+taxifolin (40 μM). “Composition #2” is a 6-compound composition of: zinc picolinate (40 μM)+copper sulfate (10 μM)+quercetin (40 μM)+EGCG (40 μM)+taxifolin (40 μM)+naringenin (40 μM). “Composition #3” is a 6-compound composition of: zinc picolinate (40 μM)+copper sulfate (10 μM)+quercetin (40 μM)+EGCG (40 μM)+taxifolin (40 μM)+berberine (40 μM).

### DETAILED DESCRIPTION OF THE INVENTION

The present invention is directed to compositions comprising unique combinations of ingredients that exhibit an anti-viral activity.

The compositions of the present invention are based on combinations of phytochemicals, including polyphenol zinc ionophores (e.g. flavonoid zinc ionophores), preferably also in combination with at least one zinc compound and at least one copper compound. The compositions preferably further comprise additional ingredients to support and/or enhance the anti-viral effect of the composition, for example, at least one peroxisome proliferator-activated receptor (PPAR) agonist such as naringenin and berberine.

Ingredients of the compositions of the present invention may be artificially synthesized. Alternatively or additionally, ingredients which are found in natural sources, such as plant or animal sources, may be extracted or purified from the natural sources. The present invention encompasses artificially synthesized compounds as well as compounds extracted and/or purified from natural sources.

In some embodiments, there is provided herein a composition for human consumption comprising: (a) at least two flavonoid zinc ionophores selected from the group consisting of: taxifolin, quercetin and epigallocatechin gallate (EGCG); (b) at least one zinc compound; and (c) at least one copper compound, optionally further comprising at least one phenol peroxisome proliferator-activated receptor (PPAR) agonist.

In some embodiments, there is provided herein an antiviral combination comprising: (a) at least two flavonoid zinc ionophores selected from the group consisting of: taxifolin, quercetin and epigallocatechin gallate (EGCG); (b) at least one zinc compound; and (c) at least one copper compound, optionally further comprising at least one phenol peroxisome proliferator-activated receptor (PPAR) agonist.

In some embodiments, there is provided herein an antiviral pack or kit comprising: (a) at least two flavonoid zinc ionophores selected from the group consisting of: taxifolin, quercetin and epigallocatechin gallate (EGCG); (b) at least one zinc compound; and (c) at least one copper compound, optionally further comprising at least one phenol peroxisome proliferator-activated receptor (PPAR) agonist, further comprising instructions for use of the pack or kit. In some embodiments, the instructions are instructions for use of the pack or kit in the prevention or treatment of a viral infection. In some embodiments, the components of the antiviral pack or kit are provided as a single composition with one or more pharmaceutically acceptable excipients. In additional embodiments, the components are provided as a plurality of compositions, for example, of each component, formulated with pharmaceutically acceptable excipients.

In some embodiments, the composition, combination, package or kit further comprises at least one additional ingredient selected from: one or more carotenoid; one or more mineral (other than zinc and copper) or a salt thereof acceptable for human consumption; one or more vitamin selected from vitamin A, vitamin C, Vitamin D, vitamin E and vitamin K2, or derivatives or salts thereof acceptable for human consumption; thymus extract; and β-sitosterol. Each possibility represents a separate embodiment of the present invention.

In additional embodiments, there is provided herein a composition for human consumption comprising at least one polyphenol zinc ionophore, optionally in combination with at least one zinc compound, and further comprising at least one additional ingredient selected from:
(i) one or more phenol peroxisome proliferator-activated receptor (PPAR) agonist;
(ii) one or more carotenoid;
(iii) one or more mineral or a salt thereof acceptable for human consumption;
(vi) one or more vitamin selected from vitamin A, vitamin C, Vitamin D, vitamin E and vitamin K2, or derivatives or salts thereof acceptable for human consumption;
(v) thymus extract; and
(vi) β-sitosterol.

In some embodiments, the composition comprises a plurality of polyphenol zinc ionophores. As used herein, a “plurality” indicates at least two. In some embodiments, the composition comprises two zinc ionophores. In additional embodiments, the composition comprises three zinc ionophores.

In some embodiments, the zinc ionophores are polyphenol zinc ionophores. In some embodiments, the zinc ionophores are flavonoid zinc ionophores. In some particular embodiments, the zinc ionophores are selected from the group consisting of EGCG, quercetin and taxifolin. In some particular embodiments, the polyphenolic zinc ionophores are EGCG and quercetin. In additional particular embodiments, the polyphenolic zinc ionophores are EGCG and taxifolin. In additional particular embodiments, the polyphenolic zinc ionophores are quercetin and taxifolin.

In some embodiments, the one or more polyphenolic PPAR agonist in the compositions according to the present invention is selected from the group consisting of naringenin, berberine, resveratrol, chlorogenic acid and baicalein. Each possibility represents a separate embodiment of the present invention. In some embodiments, a composition according to the present invention comprises a plurality of polyphenol PPARx agonists. For example, the composition may include two, three, four, or five polyphenol PPAR agonists. Each possibility represents a separate embodiment of the present invention.

In some embodiments, a composition according to the present invention comprises at least one zinc compound. In some embodiments, the composition comprises a single zinc compound. In other embodiments, the composition comprises a plurality of zinc compounds. In some embodiments, the at least one zinc compound is selected from the group consisting of zinc picolinate, zinc citrate, zinc sulfate, zinc bisglycinate, zinc gluconate, zinc acetate, zinc orotate, zinc oxide, zinc-carnosine, zinc chloride, zinc carbonate, zinc ammoniate, zinc lactate, zinc stearate, zinc ascorbate, zinc-histidine complex, zinc-methionine complex, zinc-lysine-complex and zinc lacto-gluconate. Each possibility represents a separate embodiment of the present invention.

In some embodiments, the one or more carotenoid is selected from phytoene and phytofluene. In some particular embodiments, the composition comprises both phytoene and phytofluene. In additional embodiments, the one or more carotenoid is bacterioruberin.

In additional embodiments, the one or more carotenoid is selected from a-carotene, (3-carotene and lycopene. Each possibility represents a separate embodiment of the present invention. In some embodiments, the composition comprises a plurality of carotenoids. For example, the composition may include two or three carotenoids. Each possibility represents a separate embodiment of the present invention.

In some embodiments, the one or more mineral is selected from zinc, copper, magnesium and selenium, or salts thereof acceptable for human consumption. In some embodiments, the composition comprises zinc and one or more additional mineral other than zinc) selected from copper, magnesium and selenium, or salts thereof acceptable for human consumption. Each of the aforementioned possibilities represents a separate embodiment of the present invention. In some particular embodiments, the one or more mineral is Se-methyl-L-selenocysteine.

In some embodiments, a composition according to the present invention comprises a plurality of minerals or salts thereof acceptable for human consumption. For example, the composition may include two or three minerals, or salts thereof acceptable for human consumption. Each possibility represents a separate embodiment of the present invention. In some embodiments, the composition comprises the one or more minerals or salts thereof acceptable for human consumption in addition to zinc.

In some embodiments, a composition according to the present invention comprises one or more vitamin selected from vitamin A, vitamin C, Vitamin D, vitamin E and vitamin K2, or derivatives or salts thereof acceptable for human consumption. It is to be understood that the term “vitamin” as used herein encompasses natural forms as well as synthetic forms and derivatives which have the biological activity of the respective vitamin.

In some embodiments, the vitamin E is selected from α,β, γ, and δ tocopherols; and α,β, γ, and δ tocotrienols. The tocopherols can exist in different isomeric forms. Pure stereoisomers or mixtures of stereoisomers may be used in compositions according to the invention. The term “vitamin E” as used herein includes natural vitamin E, as well as derivatives thereof which have biological vitamin E activity, e.g. carboxylic acid esters, such as vitamin E acetate, propionate, butyrate or succinate. In some exemplary embodiments, the vitamin E derivative is alpha-tocopheryl succinate. Each of the aforementioned possibilities represents a separate embodiment of the present invention.

In some embodiments, the vitamin K2 or derivative thereof is selected from menaquinone-4 (MK-4), menaquinone-7 (MK-7), menaquinone-6 (MK-6) and menaquinone-9 (MK-9). Each possibility represents a separate embodiment of the present invention.

In some embodiments, a composition according to the present invention is devoid of menthol.

In some embodiments, a composition according to the present invention is devoid of hydroxyethylrutosides. In some embodiments, a composition according to the present invention is devoid of troxerutin.

In some embodiments, the composition comprises: at least one flavonoid zinc ionophore selected from EGCG, quercetin and taxifolin, optionally in combination with at least one zinc compound; and one or more polyphenol PPAR agonist selected from naringenin, berberine, resveratrol, chlorogenic acid and baicalein. Each possibility represents a separate embodiment of the present invention.

In some embodiments, the composition comprises: at least two flavonoid zinc ionophores selected from EGCG, quercetin and taxifolin, optionally in combination with at least one zinc compound; and one or more polyphenol PPAR agonist selected from naringenin, berberine, resveratrol, chlorogenic acid and baicalein. Each possibility represents a separate embodiment of the present invention.

In some particular embodiments, the composition comprises: EGCG, quercetin and at least one zinc compound, and further comprising one or more polyphenol PPAR agonist selected from naringenin, berberine, resveratrol, chlorogenic acid and baicalein. Each possibility represents a separate embodiment of the present invention.

In additional particular embodiments, the composition comprises: EGCG, taxifolin and at least one zinc compound, and further comprising one or more polyphenol PPAR agonist selected from naringenin, berberine, resveratrol, chlorogenic acid and baicalein. Each possibility represents a separate embodiment of the present invention.

In some embodiments, the composition comprises at least one flavonoid zinc ionophores selected from EGCG, quercetin and taxifolin, optionally in combination with at least one zinc compound; and one or more carotenoid.

In some embodiments, the composition comprises at least one flavonoid zinc ionophores selected from EGCG, quercetin and taxifolin, optionally in combination with at least one zinc compound; and one or more mineral or a salt thereof acceptable for human consumption.

In some embodiments, the composition comprises at least one flavonoid zinc ionophores selected from EGCG, quercetin and taxifolin, optionally in combination with at least one zinc compound; and one or more vitamin selected from vitamin A, vitamin C, vitamin D, vitamin E and vitamin K2, or derivatives or salts thereof acceptable for human consumption.

In some embodiments, the composition comprises: the flavonoid zinc ionophores taxifolin, quercetin and EGCG; a zinc compound; and a copper compound. In some embodiments, the composition further comprises naringenin. In some embodiments, the composition further comprises berberine.

In some embodiments, the composition comprises: taxifolin, quercetin and EGCG; a zinc compound; and a copper compound. In some embodiments, the composition comprises: taxifolin, quercetin and EGCG; zinc picolinate; and copper sulfate.

In some embodiments, the composition comprises: taxifolin, quercetin and EGCG; a zinc compound; a copper compound; and naringenin. In some embodiments, the composition comprises: taxifolin, quercetin and EGCG; zinc picolinate; copper sulfate; and naringenin.

In some embodiments, the composition comprises: taxifolin, quercetin and EGCG; a zinc compound; a copper compound; and berberine. In some embodiments, the composition comprises: taxifolin, quercetin and EGCG; zinc picolinate; copper sulfate; and berberine.

In some embodiments, the composition comprises: taxifolin, quercetin and EGCG; a zinc compound; a copper compound; naringenin and berberine. In some embodiments, the composition comprises: taxifolin, quercetin and EGCG; zinc picolinate; copper sulfate; naringenin and berberine.

In some embodiments, the composition comprises: the flavonoid zinc ionophores taxifolin, quercetin and EGCG; a zinc compound; a copper compound; naringenin;

berberine; a selenium compound; vitamin A and Vitamin D3.

In some particular embodiments, the composition comprises: taxifolin, quercetin and EGCG; zinc picolinate; copper bisglycinate chelate; naringenin; berberine; Se-Methyl-L-selenocysteine; vitamin A and Vitamin D3.

The term “comprising” may also encompass the meaning of “consisting of” and “consisting essentially of”, and may be substituted by these terms.

Zinc is a chemical element and an essential mineral with a normal oxidation state of (+2) (Zn²⁺). Zinc is required in humans for the function of over 300 enzymes and around 1,000 transcription factors. Zinc deficiency affects about two billion people worldwide and is associated with many diseases. Zinc deficiency is usually due to insufficient dietary intake, and can be associated with malabsorption, acrodermatitis enteropathica, chronic liver disease, chronic renal disease, sickle cell disease, diabetes, malignancy, and other chronic illnesses. An ionophore is a chemical species that reversibly binds ions. Many ionophores are lipid-soluble entities that transport ions across the cell membrane and enable ions to penetrate into cells. Zinc ionophores transport extracellular zinc across a cell membrane. The present invention utilizes non-toxic polyphenols, particularly flavonoids, which can transport zinc cations through the cell membrane. Without being bound by any particular theory of a mechanism of action, it is contemplated that administering a zinc compound together with flavonoid zinc ionophores (such as EGCG, quercetin and taxifolin) increases intracellular zinc content and intracellular zinc concentration. Zn′ ions possess antiviral activities toward viral pathogenesis processes and were shown to inhibit viral entry, intracellular replication, and spread to other cells, tissues, and organs during the pathogenesis of viral diseases.

In some embodiments, the compositions of the present invention comprise at least one zinc compound. A composition according to the present invention may include zinc compounds at doses ranging, for example, from: 5-15 mg, 15-30 mg, 30-60 mg, 60-120 mg of elemental zinc, including each value within the range. Each possibility represents a separate embodiment of the present invention.

Copper (“Cu”) is an essential trace mineral that occurs in all body tissues and is necessary for a range of bodily functions including for the immune system functioning. In some embodiments, the compositions of the present invention comprise at least one copper compound. In some embodiments, a composition according to the present invention comprises a single copper compound. In other embodiments, a composition according to the present invention comprises a plurality of copper compounds. In some embodiments, the at least one copper compound is selected from the group consisting of copper sulfate, copper citrate, copper picolinate, copper gluconate, copper oxide, copper glycinate, copper bisglycinate and copper amino acid chelates. Each possibility represents a separate embodiment of the present invention.

A composition according to the present □nventtion may include copper compounds at doses ranging, for example, from: 0.25-4 mg, 0.25-1 mg, 0.25-2, 1-2 mg, 2-4 mg of elemental copper, including each value within the range. Each possibility represents a separate embodiment of the present invention.

A composition according to the present invention may include polyphenols, each at a dose ranging, for example, from: 10-40 mg, 40-100 mg, 100-400 mg, 400-1,000 mg, including each value within the range. Each possibility represents a separate embodiment of the present invention.

Flavonoids are a sub-group of polyphenolic plant metabolites, found in a variety of fruits and vegetables. Flavonoids are classified into subclasses based on chemical structures, six of which, namely anthocyanidins, flavanols (also referred to as flavan-3-ols), flavonols, flavones, flavanones and isoflavones, are the main subclasses with dietary and health significance. Flavonoids' structure and classification is reviewed, for example, in Panche et al. (2016) J Nutr Sci., 5: e47.

The compositions of the present invention comprise dietary flavonoids which are zinc ionophores. In some embodiments, a composition according to the present invention comprises a plurality of flavonoid zinc ionophores, at a 1:1 molar ratio.

In some embodiments, the flavonoid zinc ionophores of the present invention are selected from EGCG, quercetin and taxifolin.

EGCG (Epigallocatechin-3-gallate), identified by CAS Registry No. 989-51-5, is a flavan-3-ol, and is the main polyphenol in green tea for example. EGCG has shown various health promoting effects acting through different pathways, for example as antioxidant and anti-inflammatory agent.

Quercetin, identified by CAS Registry No. 117-39-5, is a flavonol present in many common foods, such as onions, apples, grapes, and berries. Quercetin is associated with antioxidant and anti-inflammatory effects.

Taxifolin, also known as dihydroquercetin, identified by CAS Registry No. 480-18-2, belongs to the subclass flavanonols in the flavonoids.

Flavonoids for use with the present invention are available commercially.

In some embodiments, the compositions of the present invention further contain additional ingredients to support and/or enhance the anti-viral effect of the composition.

PPARx agonists act upon at least part of the peroxisome proliferator-activated receptors. PPAR agonists according to the present invention encompasses compounds that induce the activity and/or expression of PPARs. PPAR agonists for use according to the present invention include polyphenols that act as PPAR agonists. For example, PPAR agonists for use according to the present invention include naringenin, naringin, mangiferin, berberine, resveratrol, chlorogenic acid and baicalein. Each possibility represents a separate embodiment of the present invention. In some particular embodiments, PPAR agonists for use according to the present invention are PPAR-gamma agonists, e.g., naringenin. In additional particular embodiments, PPAR agonists for use according to the present invention are PPAR-alpha agonists, e.g., berberine.

A composition according to the present invention may include PPAR agonists, each at doses ranging, for example, from: 10-50 mg, 50-100 mg, 50-200 mg, 100-200, 100-500, 100-1000 mg, including each value within the range. Each possibility represents a separate embodiment of the present invention.

Naringenin, identified by CAS Registry No. 480-41-1, is a type of flavonoid found abundantly in grapefruit and in other variety of fruits and herbs. Naringenin was found to inhibit the assembly and long-term production of infectious hepatitis C virus particles through a PPAR-mediated mechanism. Also, naringenin impaired the in vitro infection of human cells by Zika virus.

Naringin, identified by CAS Registry No. 10236-47-2, is a flavanone glycoside that occurs naturally in citrus fruits, especially in grapefruit, where naringin is responsible for the fruit's bitter taste. Naringin was found to display strong anti-inflammatory and antioxidant activities.

Mangiferin, identified by CAS Registry No. 4773-96-0, is a bioactive ingredient predominantly isolated from the mango tree, with potent antioxidant activity and multifactorial pharmacological effects, including antidiabetic, antitumor, lipometabolism regulating, cardioprotective, anti-hyperuricemic, neuroprotective, antioxidant, anti-inflammatory, antipyretic, analgesic, antibacterial, antiviral and immunomodulatory effects.

Berberine, identified by CAS Registry No. 2086-83-1, is a bitter compound polyphenol found in the roots of several plants including goldenseal, barberry, and Oregon grape. It was found to be a potent agonist of PPARs. It has been shown that berberine reduces virus replication and targets specific interactions between the virus and its host. It also intercalates into DNA and inhibits DNA synthesis and reverse transcriptase activity. It is also thought to inhibit penetration of the virus into cells. In addition, it inhibits replication of herpes simplex virus (HSV), human cytomegalovirus (HCMV), human papillomavirus (HPV), and human immunodeficiency virus (HIV). Also, this isoquinoline alkaloid has the ability to regulate the MEK-ERK, AMPK/mTOR, and NF-κB signaling pathways, which are necessary for viral replication. Furthermore, it has been reported that berberine supports the host immune response, thus leading to viral clearance.

Resveratrol, identified by CAS Registry No. 501-36-0, is a stilbenoid (a type of natural phenol), and a phytoalexin produced by several plants in response to injury or when the plant is under attack by pathogens, such as bacteria or fungi. Sources of resveratrol in food include the skin of grapes, blueberries, raspberries, mulberries, and peanuts. It is a natural PPARalpha/gamma ligand. Resveratrol anti-viral effect was shown on influenza virus, Epstein-Barr virus (EBV), herpes simplex virus (HSV), respiratory syncytial virus (RP SV), human immunodeficiency virus (HIV), duck enteritis virus, human metapneumovirus, African swine fever virus, human rhinovirus, cytomegalovirus and hepatitis C virus.

Chlorogenic acid, identified by CAS Registry No. 327-97-9, is the ester of caffeic acid and (—)-quinic acid, related to polyphenol family of esters. Chlorogenic acid can be found in many plants, leaves of Hibiscus sabdariffa, eggplants, peaches, prunes and coffee beans. Chlorogenic acid is a PPARalpha/gamma agonist. It was shown to have antiviral activity against influenza A and hepatitis B.

Baicalein, identifies by CAS Registry No. 491-67-8, is a type of flavonoid, originally isolated from the roots of Scutellaria baicalensis and Scutellaria lateriflora. It is a PPAR agonist. Baicalein was found to have an antiviral activity against dengue virus and influenza virus.

Carotenoids are yellow, orange, and red organic pigments that are produced by plants and algae, as well as several bacteria, and fungi. Phytoene, identified by CAS Registry No. 13920-14-4, is an intermediate in the biosynthesis of carotenoids. Phytofluene, identified by CAS Registry No. 27664-65-9, is a colorless carotenoid found naturally in tomatoes and other vegetables. International Publication No. WO 2017/029674 describes the use of a combination of phytoene and phytofluene and compositions comprising same for delaying viral infection.

Bacterioruberin, identified by CAS Registry No. 32719-43-0, is a red-colored pigment found in several Halobacterium and Haloarcula species.

Vitamin A is a group of unsaturated nutritional organic compounds that includes retinol, retinal, and several provitamin A carotenoids (most notably beta-carotene). One of vitamin A functions is the maintenance of the immune system. It plays a role in many areas of the immune system, particularly in T cell differentiation and proliferation.

Vitamin C is a vitamin found in various foods and is available for purchase as a dietary supplement. Examples of vitamin C forms include ascorbic acid, calcium ascorbate and magnesium ascorbate. There is some evidence that regular use of supplements may reduce the duration of the common cold.

Vitamin E is a group of compounds mainly containing fat soluble compounds but also compounds which are not fat soluble. The main fat-soluble forms of vitamin E are tocopherols and tocotrienols. Vitamin E is an antioxidant found abundantly in oils and fats.

Vitamin K2 is a type of vitamin K that is both a tissue and bacterial product, and is usually found in animal products or fermented foods. The richest dietary sources of K2 are animals' livers and fermented foods, e.g. cheeses consumed in western diets and fermented soybean products.

Thymus extract is produced from the glands of cows or calves. The thymus is a gland that is involved in the regulation of the body's immune response. Thymus extract is used for infectious diseases including recurrent respiratory infections, colds, flu, H1N1 “swine” flu, hepatitis B, hepatitis C, Epstein-Barr virus (EBV), mononucleosis, herpes and shingles.

B-sitosterol, identified by CAS No. 83-46-5, is a phytosterol (plant sterol) with chemical structures similar to that of cholesterol. B-sitosterol is widely distributed in the plant kingdom and found in vegetable oil, nuts, avocados and prepared foods.

In some embodiments, the compositions of the present invention are useful for preventing or treating respiratory viral infections, namely, viral infections of the respiratory system. A respiratory viral infection refers to a viral infection that specifically affects the upper respiratory tracts (including the nose, nasal cavities, mouth, throat, voice box, and/or larynx), lower respiratory tracts (including the trachea, bronchi, and/or lungs) or both.

In some embodiments, the compositions of the present invention are useful for preventing or treating viral infections of the respiratory system caused by RNA viruses.

For example, in some embodiments, the viral infection is caused by a virus selected from the group consisting of an influenza virus (influenza A and B), a respiratory syncytial virus, a parainfluenza virus, a metapneumovirus, a rhinovirus and a coronavirus. Each possibility represents a separate embodiment of the present invention.

In additional embodiments, the compositions of the present invention are useful for preventing or treating viral infections of the respiratory system caused by DNA viruses. For example, in some embodiments, the viral infection is caused by a virus selected from the group consisting of an adenovirus and a bocaviruses. Each possibility represents a separate embodiment of the present invention.

In some embodiments, a viral infection according to the present invention is a respiratory viral infection, caused by a virus selected from the group consisting of an influenza virus, a respiratory syncytial virus, a parainfluenza viruses, a metapneumovirus, a rhinovirus, a coronavirus, an adenovirus and a bocavirus. Each possibility represents a separate embodiment of the present invention.

In some embodiments, the coronavirus is a betacoronavirus. In some embodiments, the coronavirus is selected from a Severe Acute Respiratory Syndrome (SARS-CoV), SARS-CoV-2 and Middle East Respiratory Syndrome (MERS-CoV). Each possibility represents a separate embodiment of the present invention.

In some embodiments, the coronavirus is selected from the group consisting of: HcoV-229E, HcoV-NL63, HcoV-OC43, HcoV-HKU1, MERS-CoV, SARS-CoV and SARS-CoV-2. Each possibility represents a separate embodiment of the present invention. The viruses are typically human viruses.

The terms “treatment” and “treating” of a viral infection, used herein interchangeably, refer to the diminishment, alleviation, or amelioration in the number, severity, or frequency of at least one clinical symptom or biochemical indices associated with or caused by a viral infection. Treating further refers to accomplishing one or more of the following: reducing the severity of the disease being treated, limiting the development of symptoms characteristic of the disease being treated, limiting worsening of symptoms characteristic of the disease being treated, shortening the duration of the disease being treated, improving clinical parameters of the disease being treated (e.g., improving parameters of lung imaging tests in subjects suffering from a pulmonary infection) and reducing disease mortality. Each possibility represents a separate embodiment of the present invention.

The term “preventing” a viral infection refers to attenuating or inhibiting a virus from entering, spreading and/or multiplying in a subject/into its cells. Preventing a viral infection encompasses reducing the risk of being infected with a virus.

The terms “administering” or “administration of” are used herein with a respect to a substance, such as a compound or a combination of compounds, that can be carried out using one of a variety of methods known to those skilled in the art. For example, a compound or a combination of compounds may be administered by oral, sublingual, buccal or intranasal administration. A compound or a combination of compounds may also be administered via routs such as intravenous (IV), subcutaneous (SC), intramuscular (IM) and intraperitoneal (IP), or using inhalers (including disks) and inhalation vaporizers. Each of the aforementioned possibilities represents a separate embodiment of the present invention.

The term “co-administration” encompasses administration of two or more agents in a substantially simultaneous manner, either in a single dosage form, e.g., a capsule or tablet having a fixed ratio of first and second amounts, or in multiple doses forms, for example, for each agent, formulated in a composition with suitable pharmaceutically acceptable excipient(s). The agents can be administered in a sequential manner in either order. When co-administration involves the separate administration of each agent, the agents are administered sufficiently close in time to have the desired effect. The term “substantially simultaneous manner” refers to the administration of two or more compounds with only a short time interval between them. The term also encompasses the administration of two or more compounds such that the compounds reach therapeutically effective concentrations in the blood of the treated subject substantially at the same time. In some embodiments, the time interval is in the range of from 0.5 to 60 minutes.

The compounds according to the present invention are formulated into pharmaceutical compositions or dietary supplement compositions, which include one or more pharmaceutically acceptable excipients designed to facilitate administration of the compounds to a subject, preferably a human subject. The term “pharmaceutically acceptable excipient” as used herein refers to an excipient that is compatible with pharmaceutical or dietary supplement administration and does not abrogate the beneficial therapeutic activity and properties of the compounds of the present invention. Examples of pharmaceutically acceptable excipients include, a binder, a filler, a diluent, a surfactant or emulsifier, a glidant or lubricant, a buffering or pH adjusting agent, a tonicity enhancing agent, a wetting agent, a thickening agent, a suspending agent, a preservative, an antioxidant, a solvent, a flavoring agent, a colorant, and a mixture or combination thereof. Each possibility represents a separate embodiment.

The compositions of the present invention may be formulated in any form suitable for the administration routes indicated above. Exemplary forms within the scope of the present invention include, but are not limited to a solution, a suspension, a powder, a bar, a meal replacement, gummies, chewing gum, or a spray. Each possibility represents a separate embodiment.

Determining the therapeutically effective amount is well within the capability of those skilled in the art, especially in light of the disclosure provided herein. The exact formulation, route of administration and dosage can be chosen by the individual physician in view of the patient's condition. The amount of a composition to be administered will be dependent on certain parameters of the subject being treated, for example, for prevention or treatment, weight, age, and the severity of the disease. The pharmaceutical composition may be administered as a single dose or multiple doses in a continuous or intermittent manner. The administration schedule includes once-daily, twice-daily, thrice-daily, etc. The term “intermittent” as used herein refers to stopping and starting at either regular or irregular intervals. For example, intermittent administration can be administration every day for a certain period of time or administration in cycles or administration on alternate days. Each possibility represents a separate embodiment. Typically, the subject is a mammal, preferably a human.

A pharmaceutical composition according to the present invention may be formulated as controlled or sustained release formulations allowing for extended release of the compounds over a predetermined time period. In accordance with these embodiments, the compositions may further comprise a sustained release agent.

A composition according to the present invention may be formulated with excipients that enhance the bioavailability of the compounds, particularly oral bioavailability.

A pharmaceutical composition of the present invention may be used in combination therapy with at least one other active agent for the treatment of a viral infection.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Example 1

### Combination of a Zinc Compound, EGCG and Taxifolin/Quercetin

A. Viral Infection Measurements in Lung Cells Treated with Zinc and Zinc Ionophores

Human epithelial carcinoma lung cells (A549) were used to test the efficacy of different treatments/composition combinations in preventing viral infection. A549 cells are adenocarcinomic human alveolar basal epithelial cells.

A549 cells were grown in a culture medium (DMEM F12, 10% FBS, and 1% of L-glu, pen strep, sodium pyruvate and non-essential amino acids) at 37° C. and 5% CO₂, inoculated and transferred to a 24-well plate, where they were treated with one of the following compositions (Table 1):
Zinc picolinate (ZnPico) (40 μM)+Taxifolin (Taxi) (40 μM)+Epigallocatechin gallate (EGCG) (40 μM) (column 1)
ZnPico (40 μM)+Quercetin (40 μM)+EGCG (40 μM) (column 2)
ZnPico (40 μM)+Hydroxychloroquine (HCQ) (20 μM) (column 3)
HCQ (20 μM) (column 4)
Taxifolin (40 μM) (column 5)
Medium+phosphate-buffered saline (PBS)+DMSO (column 6)

4 hours after the addition of said treatments, the cells were infected with several concentrations of an influenza virus (rows A-C) or left uninfected (row D).

TABLE 1

| | 1 | 2 | 3 | 4 | 5 | 6 |
|---|---|---|---|---|---|---|
| A | ZnPico (40 μM) + | ZnPico (40 μM) + | ZnPico (40 μM) + | HCQ | Taxi | Medium + |
| | Taxi (40 μM) + | Quercetin (40 μM) + | HCQ (20 μM) | (20 μM) | (40 μM) | PBS + |
| | EGCG (40 μM) | EGCG (40 μM) | | | | DMSO |
| B | ZnPico (40 μM) + | ZnPico (40 μM) + | ZnPico (40 μM) + | HCQ | Taxi | Medium + |
| | Taxi (40 μM) + | Quercetin (40 μM) + | HCQ (20 μM) | (20 μM) | (40 μM) | PBS + |
| | EGCG (40 μM) | EGCG (40 μM) | | | | DMSO |
| C | ZnPico (40 μM) + | ZnPico (40 μM) + | ZnPico (40 μM) + | HCQ | Taxi | Medium + |
| | Taxi (40 μM) + | Quercetin (40 μM) + | HCQ (20 μM) | (20 μM) | (40 μM) | PBS + |
| | EGCG (40 μM) | EGCG (40 μM) | | | | DMSO |
| D | ZnPico (40 μM) + | ZnPico (40 μM) + | ZnPico (40 μM) + | HCQ | Taxi | Medium + |
| | Taxi (40 μM) + | Quercetin (40 μM) + | HCQ (20 μM) | (20 μM) | (40 μM) | PBS + |
| | EGCG (40 μM) | EGCG (40 μM) | | | | DMSO |

A = infected 5 μl;
B = infected 1 μl;
C = infected 0.5 μl;
D = uninfected

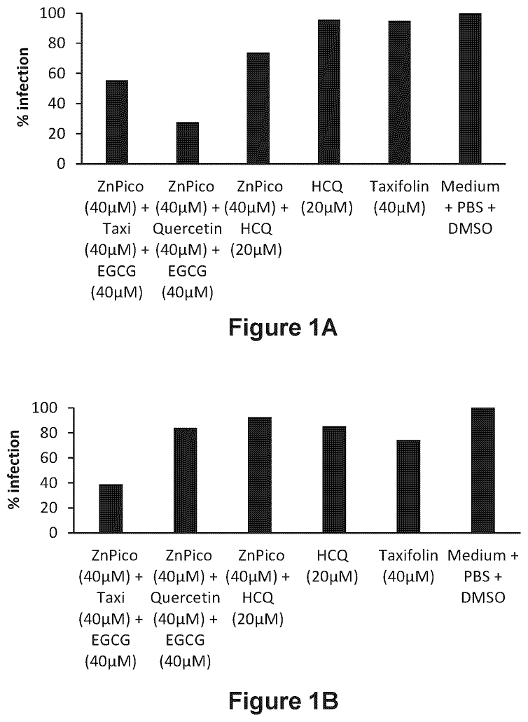

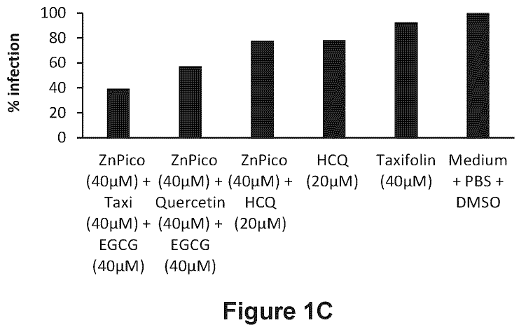

The cells were incubated in the presence of the virus for 1 hour, after which the virus-containing medium was replaced with a fresh medium containing only the indicated compositions. After additional 24 hours the cells were analyzed to quantify viral infection. The results are summarized in FIGS. 1A-1B. As can be clearly seen in FIGS. 1A-1B, both ZnPico (40 μM)+Quercetin (40 μM)+EGCG (40 μM) and ZnPico (40 μM)+Taxifolin (Taxi) (40 μM)+EGCG (40 μM) showed reduction in the percentage of cells infected with the virus. Remarkably, the combinations according to the present invention were particularly effective in preventing the viral infection of the cells, even more than ZnPico (40 μM)+HCQ (20 μM).

B. Free Zinc Quantification in Lysates of Lung Cells Treated with Zinc and Zinc Ionophores

A549 cells were grown in a culture medium (DMEM F12, 10% FBS, and 1% of L-glu, pen strep, sodium pyruvate and non-essential amino acids) at 37° C. and 5% CO₂. On the following day, the cells were treated with one of the following compositions:
ZnPico (40 μM)+Taxi (40 μM)+Epigallocatechin gallate (EGCG) (40 μM)
ZnPico (40 μM)+Quercetin (40 μM)+EGCG (40 μM)
ZnPico (40 μM)+HCQ (20 μM)
ZnPico (40 μM)+medium+DMSO
Medium+PBS+DMSO

24 hours after the addition of said treatments, cells were washed, trypsinized and lysed with an EDTA-free lysis buffer, and a free zinc quantification assay was performed using a commercial kit according to the kit supplied protocol. Briefly, cell lysates are deproteinized by adding 7% TCA (trichloroacetic acid) solution and then neutralized by adding Na₂CO₃. After the sample preparation, a zinc fluorescent indicator is added in order to measure free zinc levels.

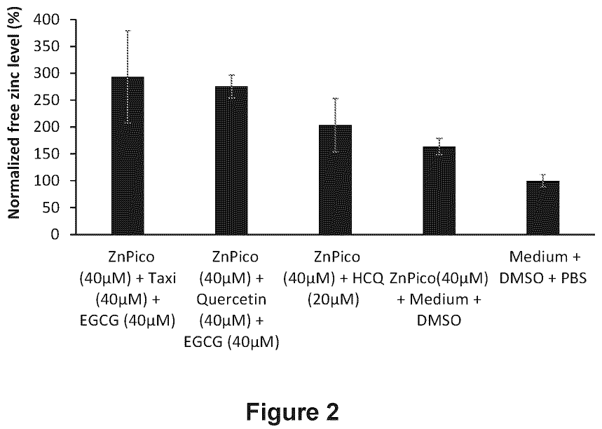

The results are summarized in FIG. 2. Free zinc levels are shown as % relative to the control (cells treated with medium+DMSO+PBS). As can be seen in FIG. 2, lysates of cells treated with either a composition comprising ZnPico (40 μM)+Taxifolin (Taxi) (40 μM)+EGCG (40 μM) or ZnPico (40 μM)+Quercetin (40 μM)+EGCG (40 μM) had higher free zinc levels compared to lysates of cells treated with ZnPico (40 μM)+HCQ (20 μM) and ZnPico (40 μM) alone.

C. Toxicity Evaluation of Cells Treated with Zinc and Zinc Ionophores

Evaluation of cytotoxicity of the compounds was carried out using MTT cell viability assay in which A549 cells were incubated in the presence of one of the following compositions for 48 hours:
ZnPico (100 μM)
ZnPico (40 μM)
ZnPico (40 μM)+HCQ (3 μM)
ZnPico (40 μM)+HCQ (6 μM)
ZnPico (40 μM)+Taxifolin (40 μM)
ZnPico (40 μM)+EGCG (40 μM)+Taxifolin (40 μM)
ZnPico (40 μM)+Quercetin (40 μM)+Taxifolin (40 μM)
ZnPico (40 μM)+Quercetin (40 μM)+EGCG (40 μM)+Taxifolin (40 μM)
Untreated cells served as a control. A further control was cells incubated with DMSO (“control+DMSO”).

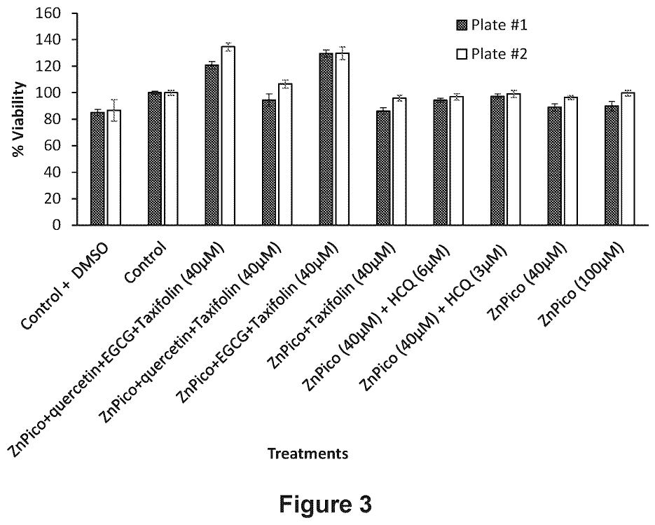

FIG. 3 summarizes the results of two repeats of the experiment. The % viability is normalized with respect to the control. Cell viability was not impaired by the different treatments, and some treatments even resulted in improved viability.

### Example 2

Combination of a Zinc Compound, a Copper Compound, Quercetin, EGCG and Taxifolin, Optionally with Naringenin

A. Viral Infection Inhibition

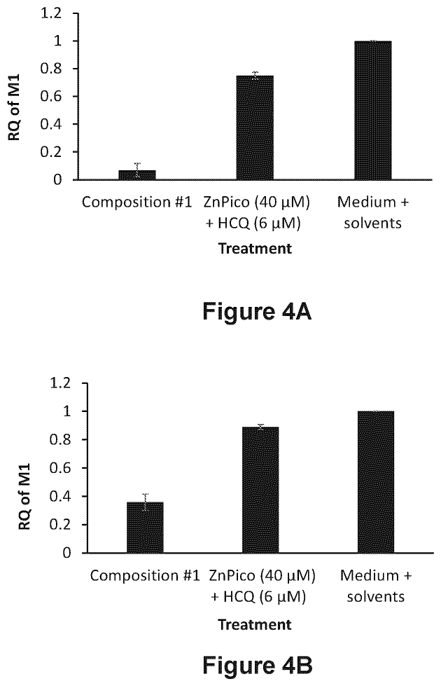

In a first set of experiments, the efficacy of different combinations in inhibiting viral infection was tested in human epithelial carcinoma lung cells (A549) and human neuroblastoma cells (SH-SYSY) infected with PR8 influenza A (H1N1) virus. The cells were grown to 90% confluency in a 24-well plate a day before infection. Four hours before infection the cells were treated with the treatments indicated in FIGS. 4A-4B. The treated cells were infected and one-hour post-infection the culture medium was changed to a fresh medium containing only the treatments without the virus. The cells were incubated for additional 24 hours and subsequently trypsinized. The detached cells were collected by centrifugation, and RNA was purified from the cells and converted to cDNA. Next, quantitative real-time PCR of the M1 viral gene was carried out to assess viral infection. A Relative Quantity (RQ) index was calculated based on the number of amplification cycles needed to exceed a required threshold, normalized with respect to a housekeeping gene (GAPDH). The RQ values were also normalized with respect to the control “Medium+solvents” that has a value of “1”.

FIGS. 4A-4B show the results in A549 cells (FIG. 4A) and SH-SY5Y cells (FIG. 4B) treated with one of the following compositions:
A 5-compound composition (Composition #1): zinc picolinate (40 μM)+copper sulfate (10 μM)+quercetin (40 μM)+EGCG (40 μM)+taxifolin (40 μM)
Zinc picolinate (40 μM)+hydroxychloroquine (HCQ) (6 μM)
Medium+solvents
Compositions' preparation:
Zinc picolinate and copper sulfate were dissolved in ultra-pure water to a stock concentration of 4 mM, quercetin and taxifolin were dissolved in DMSO to a stock concentration of 8 mM, EGCG was dissolved in DMEM cell culture medium to a stock concentration of 2 mM, and hydroxychloroquine was dissolved in PBS to a stock concentration of 4 mM. Then, the compounds were added to DMEM cell culture medium to obtain the relevant working concentrations stated above. The control “Medium+solvents” contain DMEM cell culture medium with the same volumes of solvents that were used in the compositions' preparation.

As can be seen in FIGS. 4A-4B, the composition according to the present invention showed a remarkable inhibition of viral infection, with over 85% reduction in viral load in A549 cells compared to the control group treated with medium+solvents. The composition according to the present invention was significantly more effective than a combination of zinc picolinate and HCQ.

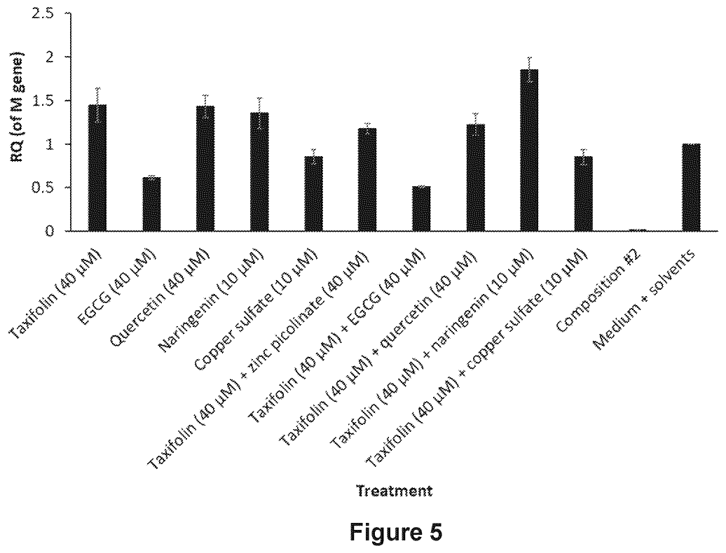

FIG. 5 shows the results in A549 cells treated with one of the following compositions:
Taxifolin (40 μM)
EGCG (40 μM)
Quercetin (40 μM)
Naringenin (10 μM)
Copper sulfate (10 μM)
Taxifolin (40 μM)+zinc picolinate (40 μM)
Taxifolin (40 μM)+EGCG (40 μM)
Taxifolin (40 μM)+quercetin (40 μM)
Taxifolin (40 μM)+naringenin (10 μM)
Taxifolin (40 μM)+copper sulfate (10 μM)
A 6-compound composition (Composition #2): zinc picolinate (40 μM)+copper sulfate (10 μM)+quercetin (40 μM)+EGCG (40 μM)+taxifolin (40 μM)+naringenin (10 μM)
Medium+solvents

The composition according to the present invention showed a remarkable inhibition of viral infection, of almost 100%, whereas each ingredient alone showed no beneficial effect or only a moderate effect. A combination of EGCG/quercetin with taxifolin was found to improve the effect achieved by each compound alone.

In a second set of experiments, the efficacy of different combinations in inhibiting viral infection was tested in A549 cells and human non-small cell lung carcinoma cells (H1299) infected with human coronavirus OC43 (HCoV-OC43). The cells were grown to 90% confluency in a 24-well plate a day before infection. Four hours before infection the cells were treated with the indicated treatments. The treated cells were infected and then incubated for additional 48 hours and subsequently trypsinized. The detached cells were collected by centrifugation, and RNA was purified from the cells and converted to cDNA. Next, quantitative real-time PCR of the ORF1 viral gene was carried out to assess viral infection. The percentage of viral gene was calculated based on the number of amplification cycles needed to exceed a required threshold, normalized with respect to a housekeeping gene (GAPDH). The percentage of the infection, measured by the percentage of the ORF1 viral gene, were also normalized with respect to the control “Medium+solvents” that has a value of 100%.

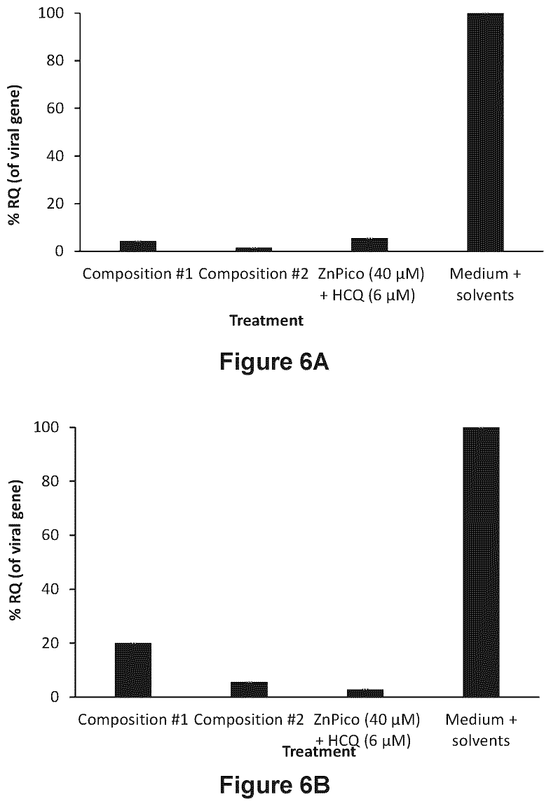

FIGS. 6A-6B show the results in A549 cells (FIG. 6A) and H1299 cells (FIG. 6B) treated with one of the following compositions:
A 5-compound composition (Composition #1): zinc picolinate (40 μM)+copper sulfate (10 μM)+quercetin (40 μM)+EGCG (40 μM)+taxifolin (40 μM)
A 6-compound composition (Composition #2): zinc picolinate (40 μM)+copper sulfate (10 μM)+quercetin (40 μM)+EGCG (40 μM)+taxifolin (40 μM)+naringenin (10 μM)
Zinc picolinate (40 μM)+hydroxychloroquine (HCQ) (6 μM)
Medium+solvents

In A549 cells, the compositions according to the present invention were remarkably effective and showed near-complete inhibition of viral infection, with Composition #2 being more effective than the composition of zinc picolinate with HCQ. In H1299 cells, the compositions according to the present invention showed a significant inhibition of viral infection, with Composition #2 being comparable to zinc picolinate with HCQ. The compositions of the present invention provide a comparable effect or even an improved effect to that of HCQ, while being significantly safer for human consumption.

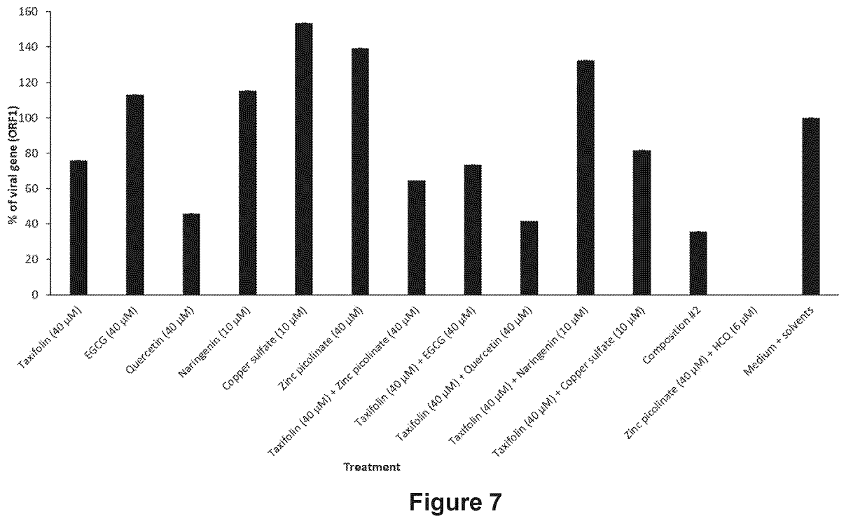

FIG. 7 shows the results in A549 cells treated with one of the following compositions:
Taxifolin (40 μM)
EGCG (40 μM)
Quercetin (40 μM)
Naringenin (10 μM)
Copper sulfate (10 μM)
Zinc picolinate (40 μM)
Taxifolin (40 μM)+zinc picolinate (40 μM)
Taxifolin (40 μM)+EGCG (40 μM)
Taxifolin (40 μM)+quercetin (40 μM)
Taxifolin (40 μM)+naringenin (10 μM)
Taxifolin (40 μM)+copper sulfate (10 μM)
A 6-compound composition (Composition #2): zinc picolinate (40 μM)+copper sulfate (10 μM)+quercetin (40 μM)+EGCG (40 μM)+taxifolin (40 μM)+naringenin (10 μM)
Zinc picolinate (40 μM)+HCQ (6 μM)
Medium+solvents

The composition according to the present invention showed significant inhibition of viral infection and was more effective than each ingredient alone. Most ingredients did not show a beneficial effect, or only showed a moderate effect. Combinations of taxifolin with each ingredient was found to improve the effect achieved by each one alone.

In a third set of experiments, the efficacy of a combination according to the present invention in inhibiting viral infection was tested in African green monkey kidney cells (Vero) infected with human metapneumovirus (hMPV). The cells were grown to 90% confluency in a 24-well plate the day before infection. Four hours before infection the cells were treated with the indicated treatments. The treated cells were infected, incubated for additional 24-hours and subsequently trypsinized. The detached cells were collected by centrifugation, and RNA was purified from the cells and converted to cDNA. Next, quantitative real-time PCR of the P viral gene was carried out to assess viral infection. A

Relative Quantity (RQ) index was calculated as described above.

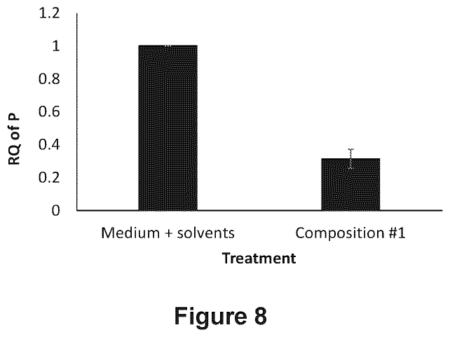

FIG. 8 shows the results in Vero cells treated with one of the following compositions:
A 5-compound composition (Composition #1): zinc picolinate (40 μM)+copper sulfate (10 μM)+quercetin (40 μM)+EGCG (40 μM)+taxifolin (40 μM)
Medium+solvents
   The composition according to the present invention showed a highly effective inhibition of viral infection.

B. Toxicity Evaluation of Cells Treated with a Combination of a Zinc Compound, a Copper Compound, Quercetin, EGCG and Taxifolin, Optionally with Naringenin

Evaluation of cytotoxicity of the compounds was carried out using MTT cell viability assay. Cells were grown to −100% confluency in a 96-well plate the day before treatment. The cells were incubated with the indicated compound or combination of compounds for 24-hours. The control cells were incubated with medium containing the solvents in which the compounds were dissolved without the addition of the compounds themselves (medium+solvents). Following incubation, MTT reagent was added for 4 hours at 37° C. Next, a detergent solution was added for another 30-min incubation at 37° C. Absorbance was determined at 570 nm and 680 nm and the results were normalized with respect to the control to obtain % of viability.

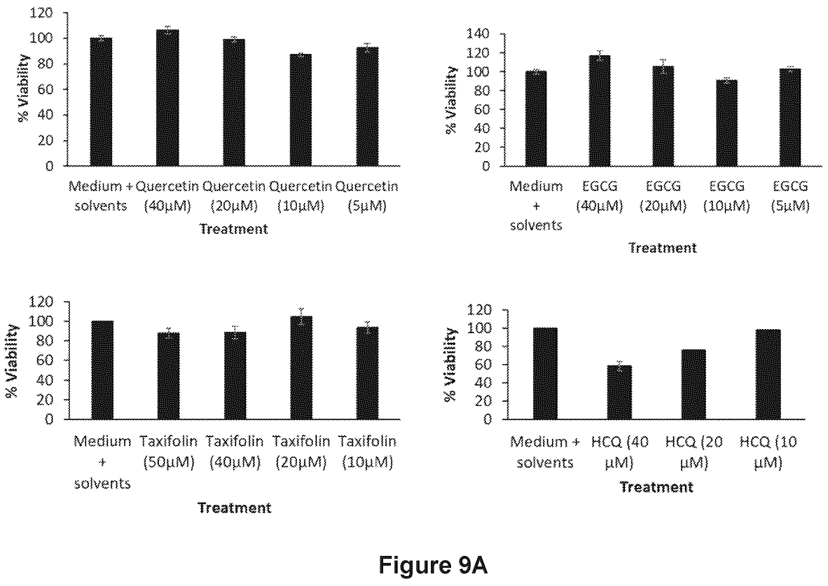

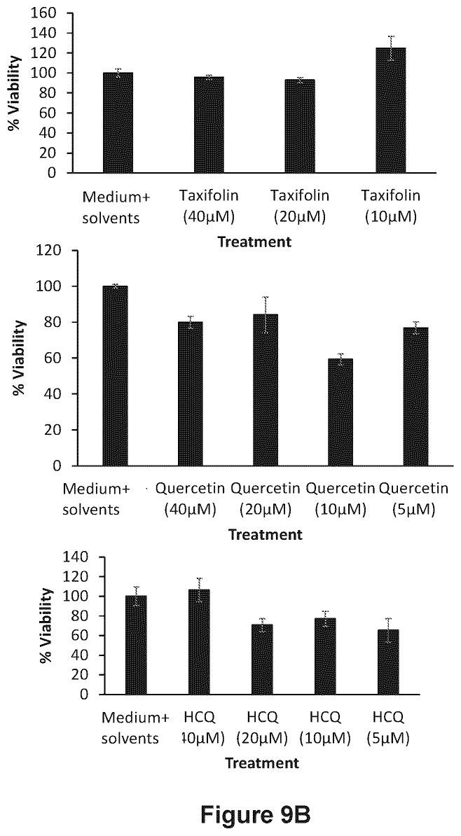

FIGS. 9A-9B show toxicity evaluation of varying doses of compounds according to the present invention in A549 cells (FIG. 9A) and H1299 cells (FIG. 9B). Toxicity evaluation of varying doses of hydroxychloroquine (HCQ) is also shown.

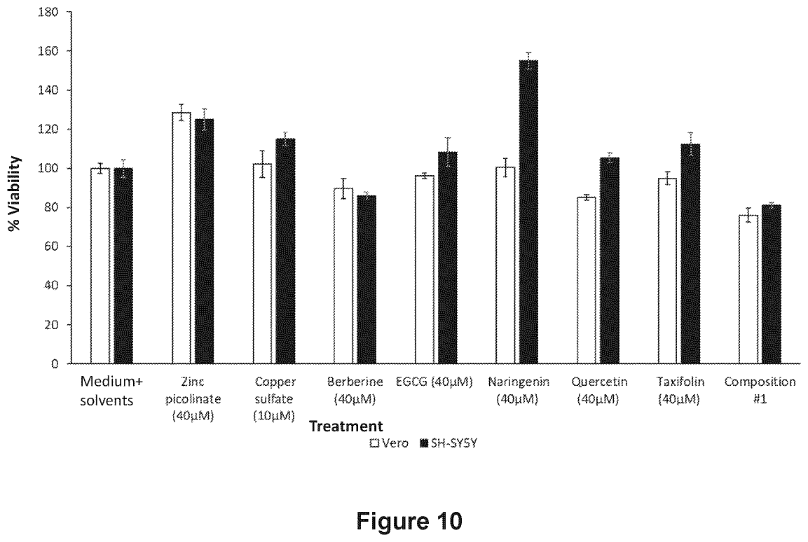

FIG. 10 shows toxicity evaluation of compounds according to the present invention and a combination according to the present invention in Vero cells and SH-SY5Y cells. Composition #1 is a 5-compound composition of: zinc picolinate (40 μM)+copper sulfate (10 μM)+quercetin (40 μM)+EGCG (40 μM)+taxifolin (40

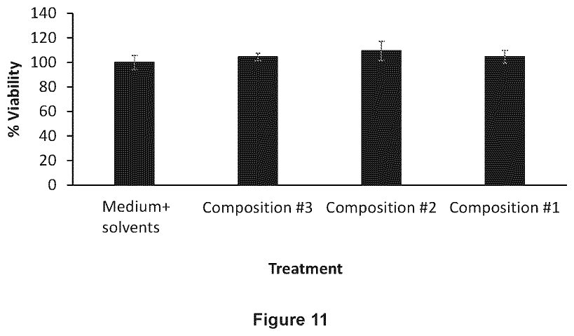

FIG. 11 shows toxicity evaluation of combinations according to the present invention in A549 cells. “Composition #”1 is a 5-compound composition of: zinc picolinate (40 μM)+copper sulfate (10 μM)+quercetin (40 μM)+EGCG (40 μM)+taxifolin (40 μM). “Composition #2” is a 6-compound composition of: zinc picolinate (40 μM)+copper sulfate (10 μM)+quercetin (40 μM)+EGCG (40 μM)+taxifolin (40 μM)+naringenin (40 “Composition #3” is a 6-compound composition of: zinc picolinate (40 μM)+copper sulfate (10 μM)+quercetin (40 μM)+EGCG (40 μM)+taxifolin (40 μM)++berberine (40 μM).

Cell viability was not impaired by the different compounds or combinations of compounds of the present invention, and some treatments even resulted in improved viability. Cell viability was maintained using much higher doses compared to HCQ.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-40. (canceled)

41. A composition for human consumption comprising:
(a) at least two flavonoid zinc ionophores selected from the group consisting of: taxifolin, quercetin and epigallocatechin gallate (EGCG);
(b) at least one zinc compound; and
(c) at least one copper compound,
optionally further comprising at least one phenol peroxisome proliferator-activated receptor (PPAR) agonist.

42. The composition of claim 41, comprising at least one phenol peroxisome proliferator-activated receptor (PPAR) agonist.

43. The composition of claim 42, wherein the at least one PPAR agonist is selected from the group consisting of naringenin, naringin, mangiferin, berberine, resveratrol, chlorogenic acid and baicalein.

44. The composition of claim 41, comprising:
taxifolin and at least one additional flavonoid zinc ionophore selected from quercetin and EGCG;
at least one zinc compound; and
at least one copper compound.

45. The composition of claim 44, comprising the flavonoid zinc ionophores taxifolin, quercetin and EGCG; a zinc compound; and a copper compound.

46. The composition of claim 45, further comprising naringenin.

47. The composition of claim 46, further comprising berberine.

48. The composition of claim 41, wherein the composition comprises: taxifolin, quercetin, EGCG, zinc picolinate and copper sulfate.

49. The composition of claim 41, wherein the composition comprises: taxifolin, quercetin, EGCG, naringenin, zinc picolinate and copper sulfate.

50. The composition of claim 41, wherein the composition is a dietary supplement composition.

51. The composition of claim 41, wherein the composition is a pharmaceutical composition.

52. A method for providing an anti-viral benefit and/or an anti-viral support to a subject, the method comprising administering to the subject a composition according to claim 41.

53. A method for providing an anti-viral benefit and/or an anti-viral support to a subject, the method comprising co-administering to the subject:
(a) at least two flavonoid zinc ionophores selected from the group consisting of: taxifolin, quercetin and epigallocatechin gallate (EGCG);
(b) at least one zinc compound; and
(c) at least one copper compound,
optionally further comprising co-administering at least one phenol peroxisome proliferator-activated receptor (PPAR) agonist selected from the group consisting of naringenin, naringin, mangiferin, berberine, resveratrol, chlorogenic acid and baicalein.

54. A method for preventing or treating a viral infection in a subject, the method comprising administering to the subject a composition according to claim 41.

55. A method for preventing or treating a viral infection in a subject, the method comprising co-administering to the subject:
(a) at least two flavonoid zinc ionophores selected from the group consisting of: taxifolin, quercetin and epigallocatechin gallate (EGCG);
(b) at least one zinc compound; and
(c) at least one copper compound,
optionally further comprising co-administering at least one phenol peroxisome proliferator-activated receptor (PPAR) agonist selected from the group consisting of naringenin, naringin, mangiferin, berberine, resveratrol, chlorogenic acid and baicalein.

56. The method of claim 52, wherein the virus is an RNA virus.

57. The method of claim 56, wherein the virus is selected from the group consisting of an influenza virus, a respiratory syncytial virus (RSV), a metapneumovirus (MPV) and a coronavirus.

58. The method of claim 54, wherein the virus is an RNA virus.

59. The method of claim 58, wherein the virus is selected from the group consisting of an influenza virus, a respiratory syncytial virus (RSV), a metapneumovirus (MPV) and a coronavirus.
